(19) United States
(12) Patent Application Publication
(10) Pub. No.: US 2024/0016964 A1
(43) Pub. Date: Jan. 18, 2024

(54) FLOOR CLEANER DOCK

(71) Applicant: Dyson Technology Limited, Wiltshire (GB)

(72) Inventor: Oliver DODGSON, Swindon (GB)

(73) Assignee: Dyson Technology Limited, Wiltshire (GB)

(21) Appl. No.: 18/265,011

(22) PCT Filed: Nov. 17, 2021
(86) PCT No.: PCT/GB2021/052975

§ 371 Date: Jun. 2, 2023

(30) Foreign Application Priority Data
Dec. 17, 2020 (GB) 2019982.4

(51) Int. Cl. A61L 2/18; A47L 9/28; A47L 9/00; A47L 11/40; A47L 11/29

(52) U.S. Cl. CPC A61L 2/18; A47L 9/2873; A47L 9/0063; A47L 11/4016; A47L 11/4083; A47L 11/29; A47L 11/4091; A47L 11/4044; A61L 2202/11

(57) ABSTRACT

A dock for a floor cleaner. For example, a dock for receiving a floor cleaner, the dock including a receiving unit for receiving the floor cleaner, and a reservoir for containing a disinfecting liquid. The dock further includes an applicator arranged to apply the disinfecting liquid to at least part of a floor cleaner received in the receiving unit. The application of disinfecting liquid to at least part of the floor cleaner may improve the hygiene of the floor cleaner and maintain the floor cleaner in a useable state.

### FIELD OF THE INVENTION

This invention relates to a dock for a floor cleaner. More particularly, but not exclusively, this invention relates to a dock for cleaning a floor cleaner.

### BACKGROUND OF THE INVENTION

Various types of floor cleaner are available. They include “dry” floor cleaners such as vacuum cleaners, and “wet” floor cleaners. Wet floor cleaners apply a cleaning fluid to the floor as part of the cleaning process. Wet floor cleaners often include a reservoir for holding cleaning fluid, means by which to apply the cleaning fluid to the floor, cleaning pads arranged to be in contact with the floor and for agitating the cleaning fluid once applied to the floor, and potentially a waste liquid reservoir for receiving waste liquid, i.e. the cleaning fluid once it has been in contact with the floor and agitated by the cleaning pads. The wet floor cleaner may also comprise at least one flexible blade, or squeegee, to help contain the cleaning fluid in the cleaning area during the cleaning process. The wet floor cleaner will also often contain a power source, for example a rechargeable battery, which powers some or all of: the application of cleaning fluid to the floor; driving the cleaning pads to agitate the cleaning fluid in contact with the floor; and removing the waste liquid into the waste liquid reservoir as required.

As such, floor cleaners, and in particular wet floor cleaners, may have several service requirements, including but not limited to: refilling cleaning fluid; emptying waste fluid; recharging the power source; conditioning the cleaning pads; and general cleaning and sanitising of the floor cleaner. The present invention seeks to address at least some of these service requirements.

### SUMMARY OF THE INVENTION

According to a first aspect, the invention provides a dock for receiving a floor cleaner, the dock comprising: a receiving unit for receiving the floor cleaner, a reservoir for containing a disinfecting liquid, and an applicator arranged to apply the disinfecting liquid to at least part of a floor cleaner received in the receiving unit.

The invention provides a convenient way to disinfect a floor cleaner during or after use. By providing a convenient way to disinfect a floor cleaner, hygiene may be improved and the potential for bad odours to develop over time may be reduced or removed. Additionally, by disinfecting the floor cleaner during or after use, it may reduce or remove the need to replace floor cleaner components that would otherwise become contaminated during use, to a point where they needed replacement.

The applicator may apply disinfecting liquid in a passive or active way. For example, in a passive way the applicator may provide a pool of disinfecting liquid into which the floor cleaner is inserted. In an active way, the applicator may squirt or spray the floor cleaner with disinfecting liquid. The applicator may comprise one or more nozzles through which the disinfecting liquid may be squirted, sprayed, or dripped in order to facilitate the application of the disinfecting liquid to the floor cleaner. The dock may comprise a pump for moving the disinfecting liquid from the reservoir for containing disinfecting liquid through the nozzles, and into contact with the floor cleaner. The nozzles may squirt or spray the disinfecting liquid with enough pressure that an agitating effect is provided by the disinfecting liquid. An agitating effect may further increase the cleaning effect of the disinfecting liquid. Alternatively or additionally, the applicator may be arranged such that the floor cleaner sits in the disinfecting liquid for some time. For example, the receiving unit may be at least partially filled with disinfecting liquid such that the parts of the floor cleaner received in the receiving unit sit in the disinfecting liquid. Such an arrangement may allow the disinfecting effect to increase over time due to the constant presence of the disinfecting liquid.

The disinfecting liquid may be aqueous hydrogen peroxide. The dock may comprise a cell arranged to create a disinfecting liquid from water. For example, a cell may be arranged to create hydrogen peroxide from water by the application of electricity. Creating a disinfecting liquid from water may reduce the running costs of the floor cleaner, may remove the need for a user to store separate cleaning chemicals, and/or may be preferable to a user having to handle disinfectant chemicals. The cell arranged to create a disinfecting liquid from water may be arranged to include the reservoir for containing the disinfecting liquid, such that the conversion of water into the disinfecting liquid takes place within the reservoir. Such an arrangement may reduce the space requirements of the dock components. Alternatively, the conversion of water into a disinfecting liquid may take place separately, after which the disinfecting liquid is transmitted into the reservoir for containing disinfecting liquid. The disinfecting liquid may be produced by the application of a concentrated disinfectant to water.

The floor cleaner may be a wet floor cleaner. The floor cleaner may comprise a cleaning pad, and the applicator may be arranged to apply the disinfecting liquid to the cleaning pad when the floor cleaner is received in the receiving unit. Application of the disinfecting liquid to the cleaning pad may improve the hygiene and/or cleaning performance of the cleaning pad. Application of the disinfecting liquid to the cleaning pad may improve the lifespan of the cleaning pad, and reduce the number of cleaning pad replacements during use of the floor cleaner.

The floor cleaner may comprise a reservoir for containing waste liquid, and the applicator may be arranged to apply the disinfecting liquid to the reservoir for containing waste liquid when the floor cleaner is received in the receiving unit. The floor cleaner may comprise a passage through which waste liquid is arranged to pass, and the applicator may be arranged to apply the disinfecting liquid to the passage through which waste liquid is arranged to pass when the floor cleaner is received in the receiving unit. Applying disinfecting liquid to a reservoir for containing waste liquid or passage through which waste liquid passes may improve the hygiene of the floor cleaner, and may help prevent bad odours developing over time.

The dock may comprise a charging unit, the charging unit arranged to charge a battery of a floor cleaner received in the receiving unit.

The dock may comprise a waste liquid reservoir for receiving waste liquid from a floor cleaner received in the receiving unit. Such an arrangement may allow for the floor cleaner to be emptied of waste liquid before the disinfecting liquid is applied to the floor cleaner.

The dock may comprise a cleaning fluid reservoir for supplying cleaning fluid to a floor cleaner received in the receiving unit.

According to a second aspect, the invention provides a dock as described with reference to the first aspect of the invention, and a floor cleaner. The floor cleaner may be received in the receiving unit of the mobile dock. The floor cleaner may comprise any of the features described with respect to the floor cleaner which may be received in the receiving unit of the dock of the first aspect of the invention.

According to a third aspect, the invention provides a method of cleaning a floor cleaner, the method comprising the steps of: docking the floor cleaner with a dock as described with reference to the first aspect of the invention such that the floor cleaner is received in the receiving unit of the dock, and the applicator applying disinfecting liquid to at least part of the floor cleaner.

The method may further comprise the step of using the cell to create a disinfecting liquid from water, prior to the applicator applying the disinfecting liquid to at least part of the floor cleaner.

It will of course be appreciated that features described in relation to one aspect of the present invention may be incorporated into other aspects of the present invention. For example, the apparatus of the invention may incorporate any of the features described with reference to the method of the invention and vice versa.

### Description of the Drawings

Embodiments of the present invention will now be described by way of example only with reference to the accompanying schematic drawings of which:

FIG. 1 shows a schematic view of a floor cleaner;

FIG. 2 shows a schematic view of a floor cleaner and dock;

FIG. 3 shows a schematic representation of components of the dock shown in FIG. 2;

FIG. 4 shows a schematic cross-sectional view of a dock and floor cleaner;

FIG. 5 shows a schematic cross-sectional view of a dock and floor cleaner; and

FIG. 6 shows a schematic cross-sectional view of a dock and floor cleaner.

### DETAILED DESCRIPTION

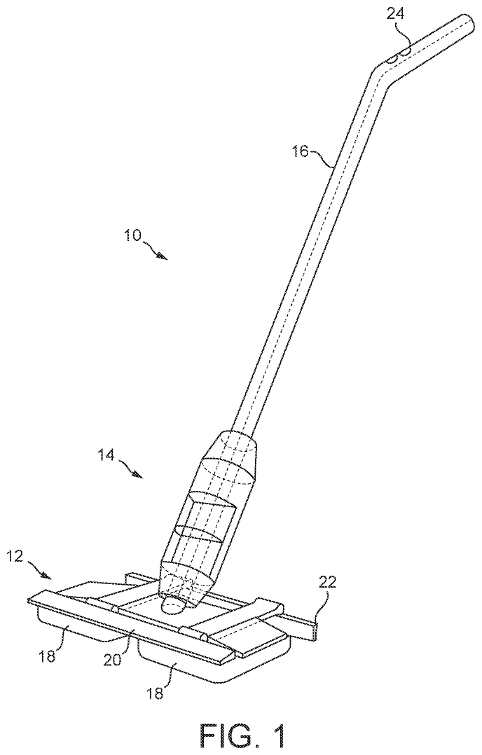

FIG. 1 shows a floor cleaner 10, which comprises a cleaning head 12, a body 14 pivotally attached to the cleaning head 12, and a handle 16 extending away from the body 14. The cleaning head 12 comprises two cleaning pads 18, which are arranged to contact a floor and oscillate during use, and a front squeegee 20 and rear squeegee 22, respectively positioned in front of and behind the cleaning pads 18. The terms “front” and “rear” will be understood by the skilled person to mean the front and rear of the cleaning head 12 when the floor cleaner 10 is being used by a user. In FIG. 1, the front squeegee 20 is shown in a retracted position, and may also be folded down into a deployed position, in which the front squeegee 20 makes contact with the floor. The body 14 comprises a cleaning fluid reservoir, a waste fluid reservoir, and a battery (not shown). The cleaning fluid reservoir is arranged to hold cleaning fluid and apply the cleaning fluid to the floor during use of the floor cleaner 10. The cleaning pads 18 oscillate during use of the floor cleaner, thereby agitating the cleaning fluid on the floor, providing a cleaning effect. The cleaning fluid becomes dirty during agitation, and the waste liquid reservoir is arranged to suck up and store the waste liquid during use of the floor cleaner. The battery is arranged to power the application of cleaning fluid to the floor, the oscillation of the cleaning pads 20, and the sucking up of the waste liquid. The handle 16 comprises a user interface 24 which turns on and off the floor cleaner functions.

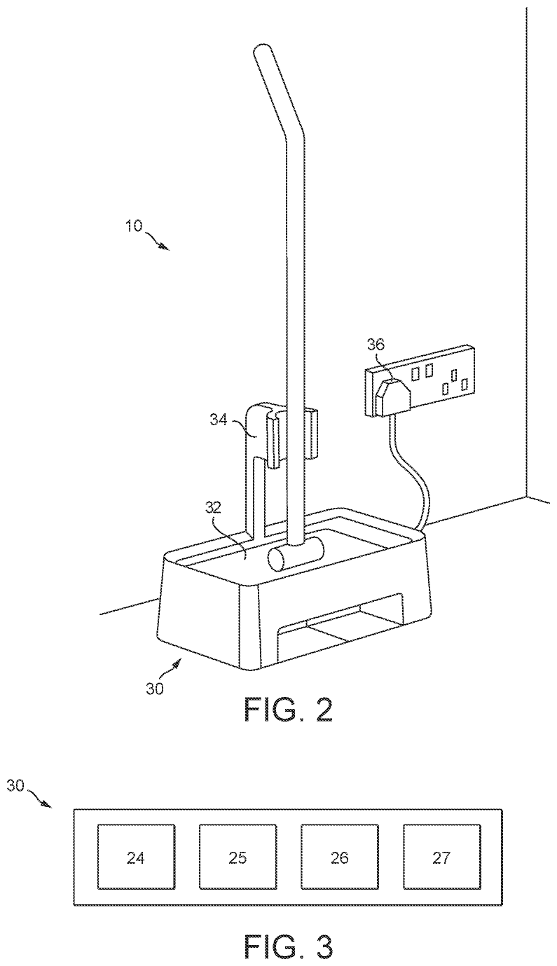

FIG. 2 shows a floor cleaner 10 and a dock 30. The dock 30 comprises a receiving unit 32 which receives the cleaning head 12 of the floor cleaner 10, and a support arm 34 against which the handle 16 of the floor cleaner 10 rests. The dock further comprises a power connection 36 to mains electricity.

FIG. 3 shows some additional components of the dock 30. In particular, the dock 30 comprises a charging unit 24 connected to mains electricity via the power connection 36. The charging unit 24 is arranged to supply charge to battery of the floor cleaner 10, either by wireless charging or by wired charging. The dock 30 also comprises a waste liquid tank 25, a cleaning fluid tank 26, and a control unit 27. The waste liquid tank 25 is arranged to receive waste liquid from the waste liquid reservoir of the floor cleaner 10. The cleaning fluid tank 26 is arranged to feed cleaning fluid into the cleaning fluid reservoir of the floor cleaner 10. The control unit 27 is arranged to control any active service functions of the dock 30, including the charging of the floor cleaner 10. Therefore, the dock 30 is able to perform several service functions on the floor cleaner 10, including charging the floor cleaner 10, emptying waste liquid from the floor cleaner 10, and refilling the floor cleaner 10 with cleaning fluid.

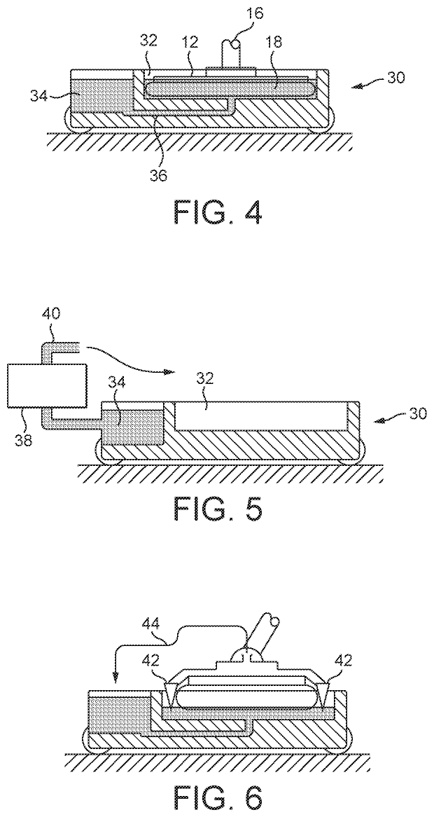

FIG. 4 shows a cross-sectional view of one possible arrangement of the dock 30 with the floor cleaner 10 received in the dock 30. The cleaning head 12 is received within the receiving unit 32, such that the cleaning pads 18 are surrounded by the receiving unit 32. The dock includes a reservoir 34 containing disinfecting liquid, the reservoir 34 located to the side of the receiving unit 32. The disinfecting liquid is hydrogen peroxide. As can be seen, the reservoir 34 is in fluid communication with the receiving unit 32 via a fluid passage 36. As such, the receiving unit also includes disinfecting liquid, and the volume of disinfecting liquid is such that the cleaning pads 18 are soaked in disinfecting liquid when the cleaning head 12 of the floor cleaner 10 is inserted into the receiving unit 32. In this way, the receiving unit 32 acts as an applicator of disinfecting liquid to the cleaning head 12. The control unit 27 is arranged to detect when the floor cleaner 10 is received in the dock 30, and initiate a timer. When a preset time is reached, the control unit 27 may indicate the preset time has elapsed, for example via an audible or visual indicator. This will ensure that the disinfecting liquid has been in contact with the floor cleaner 10 for the period of time required to ensure the disinfecting liquid provides a disinfecting effect. In an alternative arrangement, the control unit 27 is in communication with a user interface, via which the user may initiate the timer.

FIG. 5 shows a cross sectional view of another possible arrangement of the dock 30, in this case without the floor cleaner 10 shown. In this arrangement, the reservoir 34 is arranged to feed water into a cell 38, which applies electricity and oxygen to the water in order to produce hydrogen peroxide. A pump (not shown) is connected to the reservoir 34 in order to pump the water through the cell 38. The disinfecting liquid is then fed via a fluid passage 40 into the receiving unit 32, or applied to other parts of the floor cleaner 10, for example the waste liquid reservoir of the floor cleaner 10, and/or passages in the floor cleaner 10 through which waste liquid passes.

FIG. 6 shows a similar arrangement to that described with reference to FIG. 4. However, in this arrangement, the floor cleaner 10 includes squeegees 42 which are arranged to suck up liquid. When the floor cleaner 10 is being used, the squeegees 42 may suck up waste liquid from the floor. When the floor cleaner 10 is received by the dock 30 and a cleaning cycle is intiated, the squeegees 42 suck up disinfecting liquid to disinfect the parts of the floor cleaner 10 which come into contact with waste liquid during use of the floor cleaner 10. The disinfecting liquid is returned to the reservoir 34 via a fluid passage 44.

Whilst the present invention has been described and illustrated with reference to particular embodiments, it will be appreciated by those of ordinary skill in the art that the invention lends itself to many different variations not specifically illustrated herein. By way of example only, certain possible variations will now be described.

Whilst the disinfecting liquid described above is hydrogen peroxide, the skilled person will appreciate any suitable disinfecting liquid may be used.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. A dock for receiving a floor cleaner, the dock comprising:
a receiving unit for receiving the floor cleaner,
a reservoir for containing a disinfecting liquid, and
an applicator arranged to apply the disinfecting liquid to at least part of a floor cleaner received in the receiving unit.

2. The dock as claimed in claim 1, further comprising a cell arranged to create a disinfecting liquid from water.

3. The dock as claimed in claim 1, wherein the floor cleaner comprises a cleaning pad, and the applicator is arranged to apply the disinfecting liquid to the cleaning pad when the floor cleaner is received in the receiving unit.

4. The dock as claimed in claim 1, wherein the floor cleaner comprises a reservoir for containing waste liquid, and the applicator is arranged to apply the disinfecting liquid to the reservoir for containing waste liquid when the floor cleaner is received in the receiving unit.

5. The dock as claimed in claim 1, wherein the floor cleaner comprises a passage through which waste liquid is arranged to pass, and the applicator is arranged to apply the disinfecting liquid to the passage through which waste liquid is arranged to pass then the floor cleaner is received in the receiving unit.

6. The dock as claimed in claim 1 wherein the disinfecting liquid comprises aqueous hydrogen peroxide.

7. The dock as claimed in claim 1, further comprising a charging unit, the charging unit arranged to charge a battery of a floor cleaner received in the receiving unit.

8. The dock as claimed in claim 1, further comprising a waste liquid reservoir for receiving waste liquid from a floor cleaner received in the receiving unit.

9. The dock as claimed in claim 1, further comprising a cleaning fluid reservoir for supplying cleaning fluid to a floor cleaner received in the receiving unit.

10. A method of cleaning a floor cleaner, the method comprising the steps of:
docking the floor cleaner with [[a]] the dock as claimed in claim 1 such that the floor cleaner is received in the receiving unit of the dock, and
the applicator applying disinfecting liquid to at least part of the floor cleaner.

11. The method of cleaning a floor cleaner as claimed in claim 9, wherein the dock further comprises a cell arranged to create a disinfecting liquid from water, and the method further comprises the step of using the cell to create a disinfecting liquid from water, prior to the applicator applying the disinfecting liquid to at least part of the floor cleaner.
